(19) United States
(12) Patent Application Publication
(10) Pub. No.: US 2015/0125821 A1
(43) Pub. Date: May 7, 2015

(54) PROCESS FOR PRODUCING A ZIRCONIA BASED DENTAL IMPLANT

(71) Applicant: 3M INNOVATIVE PROPERTIES COMPANY, SAINT PAUL, MN (US)

(72) Inventors: Björn Theelke, Landsberg Am Lech (DE); Rainer K. Dittmann, Munchen (DE); Markus Muellritter, Tutzing (DE)

(21) Appl. No.: 14/407,769

(22) PCT Filed: Mar. 12, 2013
(86) PCT No.: PCT/US2013/030424

§ 371 Date: Dec. 12, 2014

(30) Foreign Application Priority Data
Jun. 18, 2012 (EP) 12172337.3

(51) Int. Cl. A61K 6/02; A61K 6/00; A61C 13/08; A61C 8/00; A61C 13/00

(52) U.S. Cl. CPC A61K 6/024; A61C 8/0015; A61C 13/0022; A61C 13/081; A61C 13/0006; A61K 6/007; A61C 2008/0046
USPC 433/201.1; 264/16

(57) ABSTRACT

The invention relates to a process for producing a zirconia based dental implant, the process comprising the steps. providing a zirconia based dental implant precursor having a density of below about 4 g/cm³, conducting a first surface treatment step to at least a part of the surface of the zirconia based dental implant precursor, sintering the zirconia based dental implant precursor to obtain a sintered zirconia based dental implant precursor, conducting a second surface treatment step to at least those parts of the surface of the sintered zirconia based dental implant precursor, which have been surface treated before. The invention also relates to a dental implant obtainable according to a process as described in the present text and a kit of parts comprising at least one zirconia based dental milling block suitable for being machined to a zirconia based dental implant precursor.

### FIELD OF THE INVENTION

The invention relates to a process for producing a zirconia based ceramic dental implant and to a zirconia based ceramic dental implant obtainable by such a process.

### BACKGROUND OF THE INVENTION

Dental implant surfaces are usually structured and/or roughened in order to allow a sufficient osseointegration. Structuring and high surfaces roughness may, however, decrease the fracture strength or fracture toughness of a ceramic dental implant.

For this reason most of the commercially available ceramic dental implants show relatively smooth surfaces. On these dental implants roughening is typically achieved by a shot peening process after sintering. Due to the high hardness of the sintered material only a comparably low roughness can be achieved.

Another option for achieving a rough surface is shot peening of pre-sintered zirconia based dental implants followed by a final sintering step. This process typically results in a relatively high surface roughness but also goes a long with comparably poor mechanical properties.

WO2007/090529 (Maxon Motor) describes a ceramic dental implant with a roughened surface. It is proposed to produce the implant in a sintering process and to already increase the surface roughness of the implant before the final sintering operation.

US 2008/0221681 (Trieu et al.) describes a method which may be used for introducing a residual compressive stress into a body portion of an implantable device.

US 2008/0213726 (Schlottig et al.) relates to a ceramic implant comprising a structured or porous surface, which can be obtained by a salt melt.

US 2008/0057475 (Feith) relates to a zirconia dental implant including an anchoring part and an abutment part, wherein the anchoring part and the abutment part have special sections corresponding to the individual dimensions of a patient's bone and/or gum. Said sections comprise roughened surfaces which are produced on the green compact before the latter is finally sintered.

US 2010/0081109 (Schlottig et al.) describes a dental implant and methods for the production thereof. The implant is produced at least area-wise by the aid of cold-isostatic compression, casting and/or injection molding to a green body with subsequent sintering to an implant, wherein prior to sintering the surface is changed and/or prepared such that after sintering a macroporous and/or macro-structured surface is present. Typically, the green body is modified by sand-blasting.

However, there is still a need for ceramic dental implants with improved surface roughness and fracture strength.

### SUMMARY OF THE INVENTION

It is an object of the present invention to provide a ceramic dental implant having on the one hand a sufficient surface roughness and having on the other hand a sufficient toughness or strength.

This object can be achieve by a process for producing a zirconia based dental implant, the process comprising the steps
providing a zirconia based dental implant precursor having a density of below about 4 g/cm³,
conducting a first surface treating step (e.g. sandblasting step) to at least a part of the surface of the zirconia based dental implant precursor,
sintering the zirconia based dental implant precursor to obtain a sintered zirconia based dental implant precursor,
conducting a second surface treating step (e.g. shot-peening step) to at least those parts of the surface of the sintered zirconia based dental implant precursor, which have been surface treated before.

The first surface treating step can also be described as a sandblasting step. The second surface treating step can also be described as a shot-peening step.

According to a further aspect the invention relates to a dental implant obtainable according to a process as described in the present text.

The invention is also related to a kit of parts comprising
at least one zirconia based dental milling block suitable for being machined to a zirconia based dental implant precursor,
a first surface treating medium (e.g. shot peening or sandblasting medium being characterized by at least one of the following features: material, size, shape and density),
a second surface treating medium (e.g. shot peening medium being characterized by at least one of the following features: material, size, shape and density),
optionally an instruction of use outlining how the shot peening medium should be applied.

Unless defined differently, for this description the following terms shall have the given meaning:

“Surface treatment” includes or means treatment of a surface by either shot peening or sandblasting.

“Shot peening” means a surface treatment where the surface is impacted with shot (e.g. metallic, glass, ceramic or organic particles) with force sufficient to create deformation. It is similar to sandblasting, except that it typically operates by the mechanism of plasticity rather than abrasion: each particle functions as a ball-peen hammer. In practice, this means that less material is removed by the process, and less dust created.

Devices and methods for conducting a shot peening process include air blast systems and centrifugal blast wheels. In the air blast systems, the shot peen medium is typically introduced by various methods into the path of high pressure air and accelerated through a nozzle directed at the part to be peened. The centrifugal blast wheel consists of a high speed paddle wheel. The shot medium is introduced in the center of the spinning wheel and propelled by the centrifugal force by the spinning paddles towards the part by adjusting the media entrance location, effectively timing the release of the media. Other methods include e.g. ultrasonic peening, wet peening, and laser peening.

“Sand blasting” means a surface treatment process, where the surface is at least partially abraded.

“Dental implant” means a “root” device, which is used in dentistry to support restorations that resemble a tooth or group of teeth to replace missing teeth.

Virtually all dental implants are so-called root-form endosseous implants, i.e., they appear similar to an actual tooth root (and thus possess a “root-form”) and are placed within the bone. The bone of the jaw accepts and osseointegrates with the implant. Osseointegration refers to the fusion of the implant surface with the surrounding bone.

Dental implants can be used to support a number of dental prostheses, including crowns, implant-supported bridges or dentures. They can also be used as anchorage for orthodontic tooth movement.

“Ceramic” means an inorganic non-metallic material that is typically produced by application of heat. Ceramics are usually hard and brittle and, in contrast to glasses, display an essentially purely crystalline structure.

A “zirconia based dental implant” means a dental implant the material of which is mainly comprised of zirconia (e.g. content of zirconia above about 55 or above about 60 or above about 65 or above about 70 wt.-% with respect to the weight of the dental implant).

Besides zirconia (ZrO2) as the main component, the dental implant may, however, also contain other metal oxides which may contribute to the stabilization of certain crystalline phases or to the toughness of the material.

Metal oxides like Y2O3, Ce2O3 or MgO can be used to stabilize (especially, partially stabilize) the tetragonal crystalline structure of zirconia. Those oxides are typically present in an amount below about 10 or below about 8 or below about 6 or below about 3 mol-% with respect to the weight of the zirconia based dental implant. Those dental implants are referred to as “metal oxide partially stabilized zirconia”. Widely used is for example Y2O3 which contributes to the stabilization of the tetragonal crystalline phase of zirconia (sometimes also abbreviated as Y-TZP material).

Metal oxides like Al2O3 can be used to increase the toughness of zirconia and are referred to as “alumina toughened zirconia”. The alumina content of alumina toughened zirconia is typically in a range from about 10 to about 30 wt.-%. Besides alumina, however, further metal oxides may be present, including those contributing to the stabilization of the tetragonal crystalline phase.

The term “zirconia based dental implant” comprises or refers to “metal oxide partially stabilized zirconia” and “alumina toughened zirconia”.

A zirconia based article is classified as “pre-sintered” if the dental article of framework has been treated with heat (temperature range from about 700 to about 1200° C.) for about 1 to about 4 hours to such an extent that the raw breaking resistance of the dental ceramic article or framework is within a range of about 10 to about 80 MPa or about 20 to about 50 MPa (measured according to the “punch on three ball test” (biaxial flexural strength) described in EN ISO 6872, edition March 2008, with the following modifications: diameter of steel ball: 6 mm; diameter of support circle: 14 mm; diameter of flat punch: 3.6 mm; diameter of sample disc: 25 mm, thickness of sample disc: 2 mm; no grinding and polishing of samples.).

A presintered ceramic article has typically a porous structure and its density (usually 3.0 g/cm³ for an yttrium partially stabilized ZrO₂ ceramic) is less compared to a completely sintered dental ceramic framework (usually 6.1 g/cm³ for an yttrium partially stabilized ZrO₂ ceramic or usually up to 5.4 g/cm³ for an alumina toughened zirconia with about 30 wt.-% alumina dispersed in a zirconia matrix). The average diameter of the pores can be in a range of about 50 nm to about 150 nm (corresponding to about 500 to about 1500 A). A typical average pore diameter is about 120 nm.

The terms “sintering” or “firing” are used interchangeably. The sintering temperature to be applied depends on the ceramic material chosen. For ZrO₂ based ceramics a typical sintering temperature range is about 1200° C. to about 1600° C.

Sintering typically includes the densification of a porous material to a less porous material (or a material having less cells) having a higher density, in some cases sintering may also include changes of the material phase composition (e.g., a partial conversion of an amorphous phase toward a crystalline phase).

By “dental mill blank” is meant a solid block (3-dim article) of material from which a dental article, dental workpiece, dental support structure or dental restoration can be machined. A dental mill blank may have a size of about 20 mm to about 30 mm in two dimensions, for example may have a diameter in that range, and may be of a certain length in a third dimension. A blank for making a single crown may have a length of about 15 mm to about 30 mm, and a blank for making bridges may have a length of about 40 mm to about 80 mm. A typical size of a blank as it is used for making a single unit has a diameter of about 24 mm and a length of about 19 mm. Further, a typical size of a blank as it is used for making multiple units has a diameter of about 24 mm and a length of about 58 mm. Besides the above mentioned dimensions, a dental mill blank may also have the shape of a cube, a cylinder or a cuboid. Larger mill blanks may be advantageous if more than one unit or multiple unit should be manufactured out of one blank. For these cases, the diameter or length of a cylindric or cuboid shaped mill blank may be in a range of about 90 to about 200 mm, with a thickness being in the range of about 10 to about 40 mm.

By “machining” is meant milling, grinding, cutting, carving, or shaping a material having a 3-dim. structure or shape by a machine. Milling is usually faster and more cost effective than grinding.

A “green body” means an un-sintered ceramic item which might still contain organic components.

A “particle” means a substance being a solid having a shape which can be geometrically determined. The shape can be regular or irregular. Particles can typically be analysed with respect to e.g. grain size and grain size distribution.

A “powder” means a dry, bulk solid composed of a large number of very fine particles that may flow freely when shaken or tilted.

“Density” means the ratio of mass to volume of an object. The unit of density is typically g/cm³. The density of an object can be calculated e.g. by determining its volume (e.g. by calculation or applying the Archimedes principle or method) and measuring its mass.

“Ambient conditions” mean the conditions which the inventive solution is usually subjected to during storage and handling Ambient conditions may, for example, be a pressure of about 900 to about 1100 mbar, a temperature of about −10 to about 60° C. and a relative humidity of about 10 to about 100%. In the laboratory ambient conditions are adjusted to about 23° C. and about 1013 mbar.

As used herein, “a”, “an”, “the”, “at least one” and “one or more” are used interchangeably. The terms “comprises” or “contains” and variations thereof do not have a limiting meaning where these terms appear in the description and claims. The term “comprise” or “contain” also encompasses the meanings “consisting essentially of” and “consisting of”.

Also herein, the recitations of numerical ranges by endpoints include all numbers subsumed within that range (e.g., 1 to 5 includes 1, 1.5, 2, 2.75, 3, 3.80, 4, 5, etc.).

### BRIEF DESCRIPTION OF THE DRAWINGS

FIG. 1 shows a general concept of the surface treatment of the surface of a dental implant.

FIG. 2 shows the dependency of bending strength (y-axis) in relation to surface roughness Ra (x-axis) for sintered samples and sintered samples after a second surface treatment.

FIG. 3 shows the dependency of surface phase content of crystal structures (%; y-axis) on the surface conditions (x-axis).

FIG. 4 shows the dependency of phase content (%; y-axis) in relation to peening pressure (bar; x-axis) and surface treatment medium applied.

FIG. 5 shows the dependency of bending strength (MPa; y-axis) in relation to peening pressure (bar; x-axis) for sintered samples and sintered samples after a second surface treatment.

FIG. 6 shows the dependency of surface roughness (Ra; y-axis) in relation to peening pressure (bar; x-axis) for sintered samples, sintered samples after a second surface treatment with zirconia particles and sintered samples after a second surface treatment with alumina particles.

### DETAILED DESCRIPTION

The process described in the present text facilitates the production of ceramic dental implants having on the one hand a sufficient surface roughness and on the other hand sufficient fracture strength or fracture toughness.

The invention combines a surface treatment of a ceramic dental implant precursor in a porous state and in sintered state.

In a first step the surface of a ceramic body (having a comparably low density) is surface treated (e.g. sandblasted) to generate a high surface roughness (Ra>1 μm) on at least some parts of the ceramic body.

Afterwards a sintering process is applied to consolidate the ceramic body (thereby increasing the density of the ceramic).

A second surface treatment step (e.g. shot peening step) follows. Without wishing to be bound to a particular theory, the second surface treatment step may compensate strength decrease caused by roughening the ceramic body in a low-density (e.g. pre-sintered) state.

This is in contrast to prior art references like US 2010/0081109 where the sandblasting steps are conducted before sintering.

It is assumed that due to the second surface treatment step the surface crystal phases of the zirconia based material are manipulated to induce compression stress zones in the lattice structure. These crystal phases can be analyzed by x-ray diffraction methods (XRD).

It is further assumed that compressive stress zones at the implant surface are able to compensate local tensile stresses especially at cavities created in the first surface treatment step under mechanical loading of the implant.

It was found that surfaced roughed samples to which a second surface treatment has been applied show equal or higher strength compared to samples with unstructured and/or smooth surfaces or samples the surface of which has been sandblasted only before sintering but not afterwards.

The roughness created in the first surface treatment step can be preserved to allow a sufficient osseointgration of the dental implant and the second surface treatment step increases the strength or toughness of the dental implant due to the creation of surface compression zones.

In contrast to this, it was found that high roughness or sharp structures typically lead to high local tensile stress which may cause implant failure at low loading forces (e.g. during implantation).

The process for producing a zirconia based dental implant according to the invention comprises two surface treatment steps.

The first surface treatment step is applied to the surface of a zirconia based dental implant precursor, that is, to an object which has not been finally sintered.

The density of the zirconia based dental implant precursor is typically below about 4 g/cm³ or below about 3.6 g/cm³ or below about 3.3 g/cm³.

The second surface treatment step is applied to the surface of the sintered zirconia based dental implant precursor.

The density of the sintered zirconia based dental implant is typically above about 5.0 g/cm³ or above about 6.0 g/cm³ or above about 6.05 g/cm³.

The zirconia based dental implant comprises, essentially consists of zirconia or consists of a zirconia based ceramic material.

The zirconia based material is typically a partially stabilized zirconia material.

Components which can be used to stabilize the zirconia based material comprise Y, Mg, Ce, La, Er and mixtures thereof.

With respect to zirconia, yttrium doped partially stabilized zirconia can be preferred. This material is often also referred to as Y-TZP and commercially available from e.g. Tosoh Comp., Japan.

A specific example of a yttrium doped zirconia material comprises 4.9 to 5.4 wt.-% Y2O3, 0.1 to 0.4 wt.-% Al2O3, and 95 to 94.2 wt.-% ZrO2 (often combined with traces of HfO2).

A specific example of an alumina toughened zirconia material comprises about 3 to about 6 wt.-% Y2O3, about 10 to about 30 wt.-% Al2O3, and about 87 to about 64 wt.-% ZrO2 (often combined with traces of HfO2).

The material of the zirconia based dental implant precursor can typically be characterized by at least one of or all of the following features:
Biaxial flexural bending strength: from about 10 to about 80 MPa or from about 20 to about 50 MPa according to EN ISO 6872:2008;
Density: below about 4 g/cm³ or below about 3.5 g/cm³ or below about 3.3 g/cm³;
Hardness: 20-70 HV1 (Vickers hardness);
ZrO2-content: above about 94.2 or above about 95 wt.-%;
Surface Roughness (Ra): below about 2 μm or below about 1.5 μm or below about 1.3 μm.

If desired, the density can be determined as follows: volumetric and gravimetric measurements.

If desired, the hardness can be determined as follows: hardness measurements by indentation of Vickers diamond pyramid according to DIN EN 843-4:2005.

If desired the surface roughness can be determined with a Laser Scanning Microscope according to ISO 4287:1997.

A more detailed description can be found in the Example section.

The dental implant precursor can be obtained by different methods. Suitable methods or processes include:
machining a zirconia based ceramic dental mill blank;
applying a casting or an injection-molding process;
applying a build-up technology.

A suitable dental mill blank typically comprises a pre-sintered zirconia based material, which is contained in a frame or fixed to a holding device.

Examples of dental mill blanks with or without a holding device are described e.g. in U.S. Pat. No. 6,454,568 (ESPE), U.S. Pat. No. 7,604,759 B2 (Gubler et al), WO 02/45614 (ETH Zurich), WO 01/13862 (3M). The content of these references is herewith incorporated by reference.

Suitable ceramic dental mill blanks are also commercially available from e.g. 3M ESPE under the brand LAVA™.

By casting or injection molding is meant a manufacturing process for producing parts from deformable materials. The material is typically fed into a barrel, mixed, and forced into a mold cavity where it hardens to the configuration of the cavity.

Build-up technologies, which are sometime also referred to as rapid-prototyping techniques include ink-jet printing, 3d-printing, multi-jet plotting, robo-casting, electrophoretic deposition, fused deposition modelling, laminated object manufacturing, selective laser sintering or melting, stereolithography, photostereolithography, or combinations thereof.

Those and other techniques are e.g. described in U.S. Pat. No. 5,902,441 (Bredt et al.), U.S. Pat. No. 6,322,728 (Brodkin et al.) and U.S. Pat. No. 6,955,776 (Feenstra et al.) and U.S. Pat. No. 7,086,863 (Van der Zel et al.).

The disclosure of these patents as it regards the description of rapid-prototyping techniques is herewith incorporated by reference and regarded as part of this application.

Commercially available examples of rapid-prototyping equipment which can be used include printers from ZCorp company like the printer ZPrinter™ 310 plus.

The first surface treatment step (e.g. sandblasting step) is applied to at least a part of the surface of the zirconia based dental implant precursor.

Usually it is sufficient, if the first surface treatment step is applied only to those regions or areas of the dental implant which should have a sufficient surface roughness. Those regions or areas are typically those, which are inserted into the bone of a patient later.

It is, however, also feasible that the first surface treatment step is applied to the whole surface of the zirconia based dental implant precursor.

The shot peen or sandblasting medium used for conducting the first surface treatment step can typically be characterized by at least one of or all of the following features:
comprising a material selected from inorganic material including zirconia (ZrO2), alumina (Al2O3), boron carbide (BC), boron nitride (BN), silica or quartz (SiO2), magnesia (MgO), tungsten carbide (WC), iron based material (steel, iron), non ferrous metals (aluminium, bronze, zinc) and mixtures thereof and organic material including polyvinylchloride, polystyrol, polyamide, nut shells, fruit stones, hard wood and mixtures thereof.
hardness: from above about R100 (Rockwell hardness) for organic materials determined according to ASTM D785 to above about HV 1 2500 (Vickers Hardness) for inorganic materials determined according to DIN EN 843-4;
mean particle size: from about 25 to about 250 μm or from about 50 to about 110 μm;
shape: spherical or non-spherical.

The first surface treatment step can typically be characterized by the following feature:
Pressure: at least about 1 to about 4 or from about 1.5 to about 3 bar.

A combination of the following parameters for the first surface treatment step has been proven to be beneficial for obtaining a surface roughened zirconia based dental implant precursor:
Pressure: from about 1 to about 2 bar;
Mean particle size of the shot medium: from about 50 to about 250 μm;
Material the shot medium is made of: alumina

The overall chemical composition of the zirconia based ceramic dental implant precursor has not changed.

However, depending on the surface treatment medium used, the surface of the zirconia based ceramic dental implant precursor may contain residues of the surface treatment medium.

Due to the surface treatment step applied, the surface of the zirconia based ceramic dental implant precursor has been roughened.

The first surface treatment step is typically done under conditions until a sufficient surface roughness of the pre-sintered dental implant precursor is achieved.

A surface roughness (Ra) after the first surface treatment step of at least about 1.2 μm or at least about 2 μm or at least about 4 μm or at least about 6 μm was found to be useful.

The surface roughness (Ra) after the first surface treatment step is typically utmost about 9 μm or utmost about 8 μm or utmost about 6 μm.

Thus, useful ranges for the surface roughness (Ra) after the first surface treatment step include from about 1.2 to about 6 μm or from about 2 to about 5 μm.

Without wishing to be bound to a particular theory, it is assumed that a sufficient surface roughness facilitates the healing process of the dental implant once inserted into the bone of a patient.

If the surface roughness is too low, it may be difficult to achieve a sufficient osseointegration of the dental implant into the bone of a patient.

However, if the surface roughness is too high, the strength or toughness of the dental implant is typically too low.

The zirconia based dental implant precursor can usually be characterized by at least one of or all of the following features (after having conducted a first shot peening step):
Density: from about 2.3 g/cm³ to about 4.0 g/cm³ or from about 3.5 g/cm³ to about 4.0 g/cm³;
Biaxial flexural bending strength: about 10 to about 80 MPa or about 20 to about 50 MPa (measured according to the “punch on three ball test” (biaxial flexural strength) described in EN ISO 6872:2008);
Surface roughness (Ra): above about 2 μm or above about 4 μm or above about 6 μm;
Monoclinic phase content measured in the surface region of the dental implant precursor (e.g. down to a depth of about 5 μm): 0%;
Distorted phase content measured in the surface region of the dental implant precursor (e.g. down to a depth of about 5 μm): 0%.

The zirconia based dental implant usually has a core region and a surface region.

Within the meaning of the present text, the surface region means a region extending from the surface of the zirconia based dental implant or precursor down to a depth of about 5 or about 10 or about 20 or about 50 μm.

Due to the limited penetration depth of the x-ray beam, the analysis of the phase content is typically performed down to a depth of about 5 μm.

Sintering of the surface treated zirconia based dental implant precursor is typically done under conditions suitable to obtain a densely sintered zirconia based.

A zirconia based ceramic is typically said to have been dense sintered, if the density of the zirconia based material is above about 5.0 g/cm³ or above about 6.0 g/cm³ or above about 6.05 g/cm³.

An alumina toughened zirconia ceramic is typically said to have been dense sintered, if the density of the zirconia based material is above about 4.5 g/cm³ or above about 5.2 g/cm³ or above about 5.8 g/cm³.

Sintering of the surface treated zirconia based dental implant precursor is typically done under the following conditions:
Temperature: from about 1200 to about 1600° C. or from about 1300 to about 1500° C.;
Duration: from about 1 to about 3 h;
Atmosphere: air.

Applying a sufficient high sintering temperature usually ensures that organic residues, which may be present or remain on the surface of the dental implant precursor are burnt after the surface treatment. Ovens which can be used for doing the sintering are commercially available from e.g. 3M ESPE under the brand LAVA™ Therm or LAVA™ Furnace 200.

The overall chemical composition of the zirconia based dental implant precursor has not changed after sintering, except for sinter and/or processing adjuvants which may have been used or added during the production of the zirconia based dental implant precursor and which have been burnt during the sintering.

However, if a surface treatment medium was used which cannot be burnt, the surface of the zirconia based dental implant precursor may contain residues of the surface treatment medium.

The zirconia based dental implant precursor can usually be characterized by at least one of or all of the following features (after having conducted a first surface treatment step and a sintering step):
Density: above about 5.0 g/cm³ or above about 6.0 g/cm³ or above about 6.05 g/cm³;
Biaxial flexural bending strength: from about 500 to about 700 or from about 550 to about 650 MPa (determined according to EN ISO 6872:2008); measurement was performed on discs manufactured in the same manner like a dental implant shaped implant precursor;
Surface roughness (Ra): above about 2 μm or above about 3 μm or above about 4 μm;
Monoclinic phase content in the surface region of the dental implant precursor: 0%;
Distorted phase content in the surface region of the dental implant precursor: 0%.

During the sintering step, the density of the zirconia based material is increased. This goes along with volume shrinkage of the material. During such a volume shrinkage also the surface roughness is affected, i.e. the surface roughness is typically decreased during sintering.

Thus, in order to ensure that the zirconia based dental implant precursor has a sufficient surface roughness after sintering, the surface roughess (Ra) of the dental implant precursor before sintering should be greater by about 4 μm or about 6 μm or about 9 μm.

The second surface treatment step (e.g. shot peening step) is done after the sintering step has been conducted.

However, intervening steps like cleaning or tempering can also be applied, if desired.

The second surface treatment step is typically done under conditions that the surface roughness achieved after the first surface treatment step is not completely destroyed.

The surface treatment is typically done to at least those parts of the surface of the sintered zirconia based dental implant precursor, onto which a first surface treatment step has been applied.

However, it is also possible that further sections or regions of the surface of the dental implant precursor are surface treated.

A surface roughness (Ra) after the second surface treatment step of at least about 1.5 μm or at least about 1.2 μm or at least about 1.5 μm was found to be sufficient.

The surface roughness (Ra) after the second surface treatment step is typically utmost about 8 μm or utmost about 6 μm.

Thus, useful ranges for the surface roughness (Ra) after the second surface treatment step include from about 1 to about 8 μm or from about 1.5 to about 6 μm or from about 1.5 to about 5 μm.

The shot peen medium used for conducting the second surface treatment step can typically be characterized by at least one of or all of the following features:
comprising a material selected from inorganic material including zirconia (ZrO2); alumina (Al2O3), boron carbide (BC), boron nitride (BN), silica or quartz (SiO2); magnesia (MgO), tungsten carbide (WC), mixtures thereof;
hardness: HV1≧1250;
mean particle size: from about 25 to about 260 μm or from about 40 to about 160 μm;
shape: spherical or non-spherical.

According to one embodiment, the hardness of the second surface treatment medium is higher than the hardness of the first surface treatment medium. Thus, it can be preferred if for the first surface treatment step a medium is used having a hardness from about R100 for organic materials to above about HV1 2500 for inorganic materials and if for the second surface treatment step a medium is used having a hardness from about HV1 1250 to about HV1 2500.

This can be beneficial in that the hardness of the dental implant precursor before sintering is comparably low and thus can be more easily roughened than after the sintering process.

The second shot peening step can typically be characterized by the following feature:
Pressure: at least about 4 or at least about 3 or at least about 1.5 bar or from about 1.5 to about 4 or from about 2 to about 3 bar.

For the second surface treatment step a combination of the following parameters has been proven to be beneficial for obtaining a surface roughened and surface strengthened zirconia dental implant:
Pressure: from about 2 to about 3 bar;
Mean particle size of the shot medium: from about 40 to 160 μm;
Material the surface treatment medium is made of: zirconia.

The invention is also directed to the zirconia based dental implant obtainable or obtained according to a process as described in the present text.

After the second surface treatment step has been applied, a zirconia based dental implant has been obtained.

However, further steps like cleaning, tempering, or sterilizing can be conducted, if desired.

The overall chemical composition of the zirconia based dental implant has not changed after the second surface treatment step has been conducted.

However, depending on the surface treatment medium used, the surface of the zirconia based dental implant precursor may contain residues of the surface treatment medium.

The crystal structure or phase content of the surface region of the dental implant described in the present text is usually different from the crystal structure or phase content of the core region. This can be determined by XRF methods, if desired.

Thus, the zirconia based dental implant obtainable or obtained according to a process as described in the present text differs from zirconia based dental implants described in the prior art, e.g. with respect to the crystal structure especially as regards the surface region. The surface of zirconia based dental implant which has not been surface treated after sintering typically show the crystal structure of the core region.

The zirconia based dental implant precursor described in the present text can usually be characterized by at least one of the following features (after having conducted a first surface treatment step, a sintering step and a second surface treatment step):
Density: above about 5 g/cm³ or above about 6.0 g/cm³ or above about 6.05 g/cm³;
Biaxial flexural bending strength: from about 600 to about 1500 or from about 750 to about 1000 MPa according to EN ISO 6872:2008;
Surface roughness (Ra): above about 1 μm or above about 1.5 μm or above about 2.0 μm;
Monoclinic phase content measured in the surface region (e.g. down to a depth of about 5 mm) of the dental implant precursor: about 0 to about 15% or about 1 to about 12%;
Distorted phase content measured in the surface region of the dental implant precursor (e.g. down to a depth of about 5 mm): above about 20 or above about 30%; useful ranges include from about 20 to about 60% or from about 30 to about 55%.

As already briefly outlined above, the second surface treatment step is conducted in order to modify the crystal phase(s) in the surface of the zirconia based ceramic dental implant precursor.

Also with respect to the second surface treatment step the analysis of the phase content is typically performed down to a depth of about 5 μm.

However, due to the surface treatment step applied the phase content of the material can be affected down to a depth of about 10 or about 20 or about 50 μm. Thus, the overall phase content with respect to the whole zirconia based dental implant will be different, i.e. lower than the phase content determined in a surface region down to a depth of about 5 μm.

The zirconia based dental implant or dental implant precursor has typically at least two surface sections A and B.

Surface section A is the section which is to be inserted into the bone of a patient.

Surface section A typically comprises more than about 60% or more than about 70% or more than about 80% of the surface of the whole dental implant.

Surface section B is the section which usually extends above the gum line of the patient after the dental implant has been inserted into the bone. Surface section B is typically visible after the insertion process.

Surface section B is typically the section onto which an abutment can be attached or fixed.

Surface section B typically comprises less than about 40% or less than about 30% or less than about 20% of the surface of the whole dental implant.

As it is the surface section A which comes into contact with the bone material of a patient, it is typically sufficient, if only this surface section is surface treated, i.e. where the first and second surface treatment steps are applied.

However, if desired, other surface sections or areas can be surface treated in addition. In this respect, the surface treatment includes the first surface treatment step and the second surface treatment step described in the present text.

It is also possible to treat the complete surface of the dental implant precursor, if desired.

The dental implants can be provided in a customized or individualized shape.

Customized dental implants are implants which have a shape and size in order to match to a lot of clinical situations.

Individualized dental implants are adapted to an individual patient. Those implants are typically produced based on geometric data obtained from or based on the clinical situation in the mouth of a patient.

The dental implants may also be in a one-piece or two-piece form.

According to a further embodiment, the invention is directed to a kit of parts comprising
at least one zirconia based dental milling block suitable for being machined to a zirconia based dental implant precursor;
a first surface treatment medium as described in the present text;
a second surface treatment medium as described in the present text; and
optionally an instruction of use containing information how to conduct the surface treatment steps and the sintering step.

The zirconia based ceramic of the dental milling block is typically in a green (unsintered) or in a pre-sintered state.

The material the zirconia based dental implant described in the present text is made of, does typically not contain components which may be detrimental to the patient's health. Dental implants are medical products and thus have to fulfil certain regulations.

Thus, components which are typically not present or wilfully added to the dental implant comprise, Na₂O, K₂O, SiO₂, TiO₂ and radioactive isotopes like ²³⁸U, ²²⁶Ra, ²³²Th.

The following examples are given to illustrate, but not limit, the scope of this invention.

### EXAMPLES

### Measurements

### Surface Roughness (Ra)

The surface roughness was determined with a Laser Scanning Microscope (CLSM VK-9710 Keyence) according to ISO 4287:1997.

### Biaxial Bending Strength

The biaxial bending strength was determined according to EN ISO 6872:2008 using a universal testing machine (Instron 5566-Instron) and is given in MPa.

### Phase Content

The phase content was determined by x-ray defraction (XRD) using a BrukerD8 Discover device (Bruker AXS) and the TOPAS™ software provided by the manufacturer (Bruker) applying the Rietveld analyses and using the Bragg-Brentano geometry.

The content of the following phases was determined: monoclinic and distorted (sometime also referred to as cubic2 phase).

The phase content calculated by the TOPAS™ software is given in wt.-%.

The measurement is typically performed down to a depth of 3 to 6 μm.

### SEM Photographs

If desired, SEM photographs can be made with a LEO 1530VP device (LEO Company).

### Materials/Sample Production

### Example 1

Process Step 1: Cylindrical samples were milled out of commercially available 3M ESPE Lava™ mill blanks containing pre-sintered zirconia material partially stabilized with Y₂O₃ using the commercially available milling device 3M ESPE Lava™ Mill.

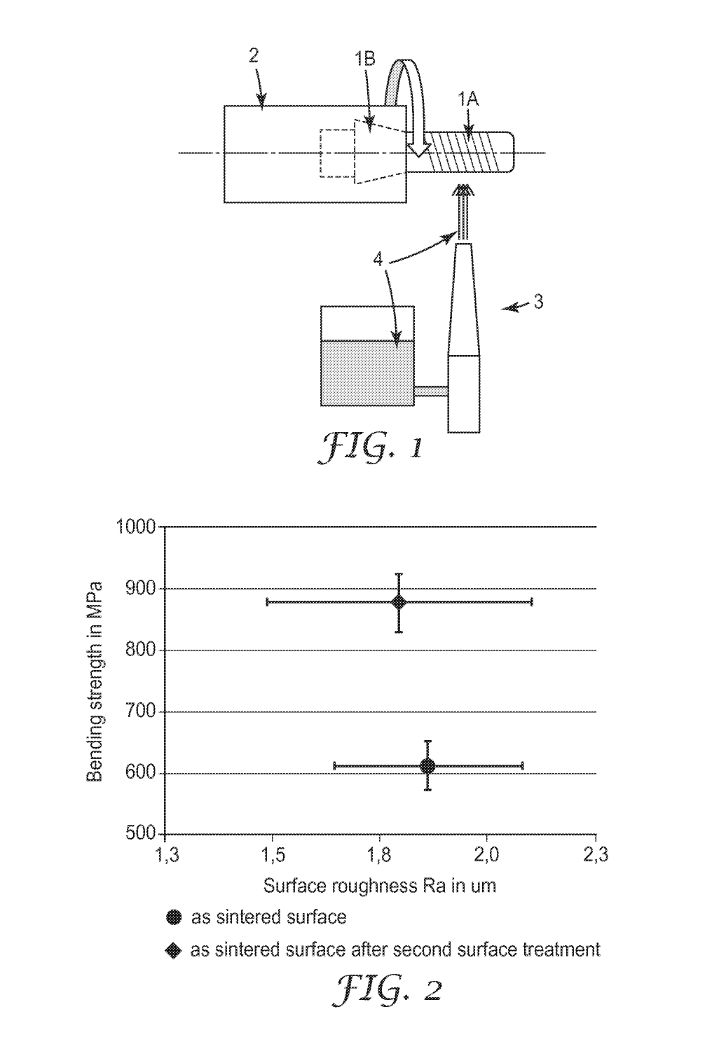

Process Step 2: In a first surface treatment step the samples were fixed to a holder and put into a surface treatment device. FIG. 1 exemplifies such an arrangement. The dental implant precursor (1) has a lower section (1A) and an upper section (1B). The dental implant precursor is fixed to a holder (2) which can be rotated along its axis. In a distance of about 5 to about 20 cm to the surface of the dental implant precursor there is a nozzle (3) through which shot peen medium particles (4) are shot onto the surface of section (1A) of the dental implant precursor. The shot peen particles (4) are stored in a container and are transported during the treatment process to the nozzle (3). The samples surfaces were treated with parameters as follows: 110 μm Alumina particles, distance to nozzle 50 mm, pressure 1.5 bar, nozzle diameter 3 mm, treatment time 5-10 s.

Process Step 3: The samples were sintered at 1500° C. for 2 hours in ambient atmosphere.

Example 1 was conducted according to the process described in US 2010/081109.

Material and surface properties were measured on disc shaped specimens treated with process steps described above. The measured values are summarized in Table 1.

TABLE 1

| Process step | 1 | 2 | 3 |
|---|---|---|---|
| Sample condition | Green or | First | As |
| | presintered | surface | sintered |
| | precursor | treatment | |
| Property | | | |
| Density in g/cm³ | 2.0-3.5 | 2.0-3.5 | >6.05 |
| Roughness Ra in μm | 0.26 | 1.86 | 1.86 |
| Fracture Strength in MPa | 30-40 | n.m. | 611 |
| Amount cubic phase in w. % | 19 | 19 | 19 |
| Amount tetragonal phase in w. % | 81 | 81 | 81 |
| Amount monoclinic phase in w. % | 0 | 0 | 0 |
| Amount distorted cubic2 phase | 0 | 0 | 0 |
| in w. % | | | |

### Example 2

The process steps 1 to 3 of Example 2 are identical to those described for Example 1.

Process Step 4: The sintered samples were treated on the roughened surface with parameters as follows: 40-160 μm Zirconia particles, distance to nozzle 50 mm, pressure 2.5 bar, nozzle diameter 3 mm, treatment time 5-10 s.

The sample properties are summarized in Table 2.

TABLE 2

| Process step | 1 | 2 | 3 | 4 |
|---|---|---|---|---|
| Sample condition | Green or | First | As | Second |
| | presintered | surface | sintered | surface |
| | precursor | treatment | | treatment |
| Property | | | | |
| Density in g/cm³ | 2.0-3.5 | 2.0-3.5 | >6.05 | >6.05 |
| Roughness Ra | 0.26 | 1.86 | 1.86 | 179 |
| in μm | | | | |
| Fracture Strength | 40 | n.m. | 611 | 876 |
| in MPa | | | | |
| Amount cubic | 19 | 19 | 19 | 11 |
| phase in w. % | | | | |
| Amount tetragonal | 81 | 81 | 81 | 38 |
| phase in w. % | | | | |
| Amount monoclinic | 0 | 0 | 0 | 6 |
| phase in w. % | | | | |
| Amount distorted | 0 | 0 | 0 | 45 |
| cubic2 phase in w. % | | | | |

### Findings

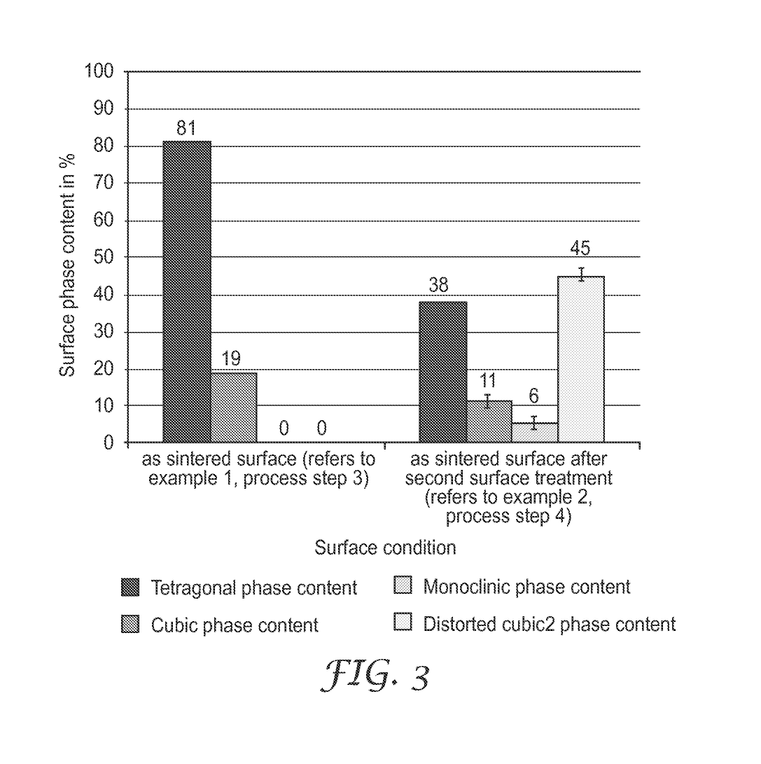

The second surface treatment (shot peening) increased the strength of roughened zirconia samples by more than about 40%. The initial roughness could be preserved during the second surface treatment by using e.g. zirconia shot, see FIG. 2.

The increase of bending strength seems to be driven by increasing the distorted (cubic2) and monoclinic phase content of the zirconia material, which seems to cause compressive stresses within the surface (depth: up to about 5 μm), see FIG. 3.

Surface treatment parameters of process step 4 were investigated in additional experiments.

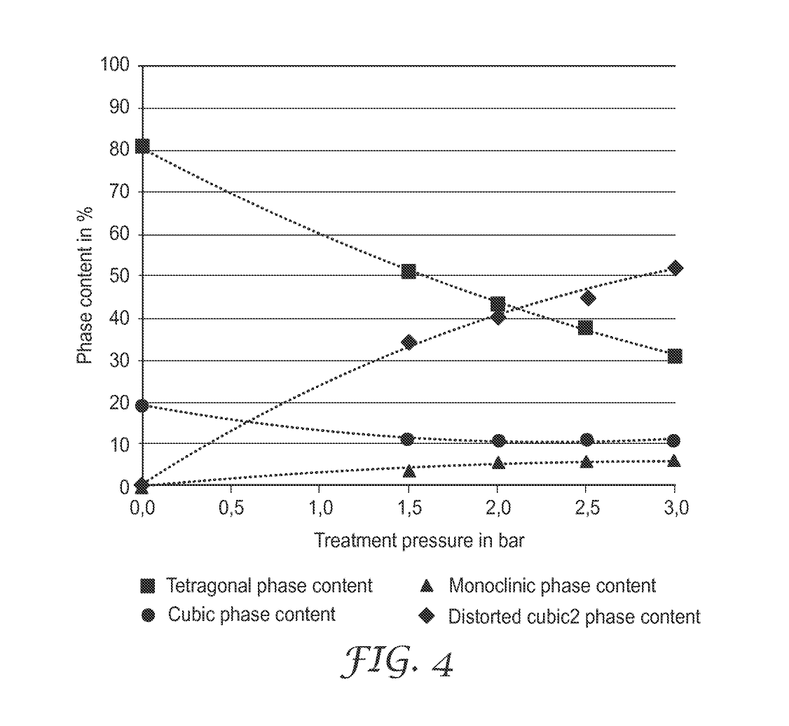

With increasing surface treatment pressure from 1.5 bar up to 3.0 bar an increase of distorted cubic2 and monoclinic phase content has been observed (see FIG. 4).

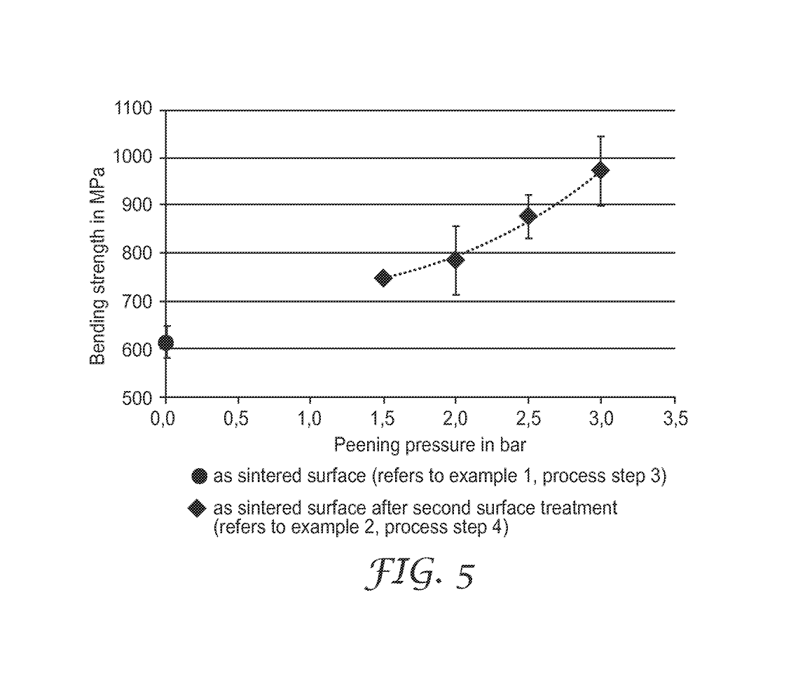

With increasing distorted surface crystal structures the material fracture strength increased within varied treatment pressure (see FIG. 5).

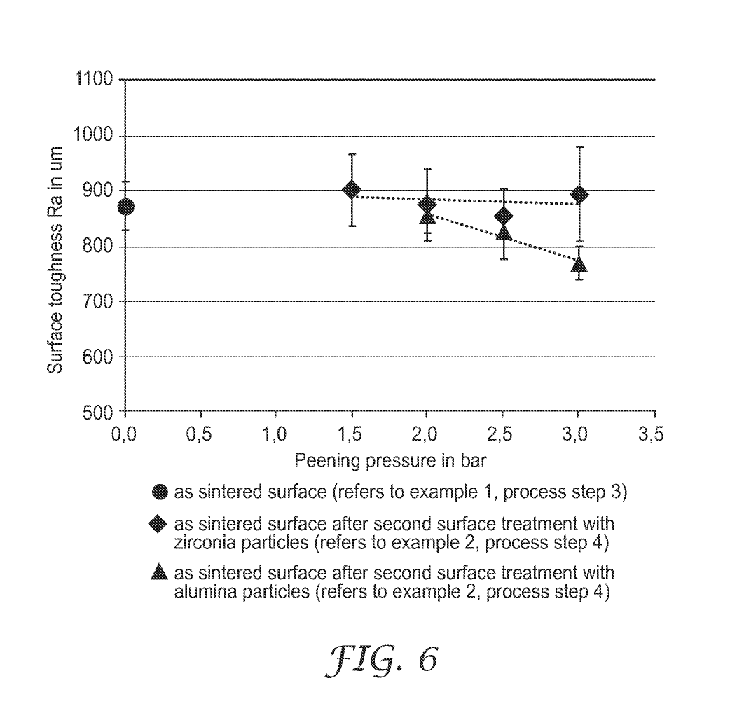

Surface treatment pressure of process step 4 was varied in a range where surface roughness created in process step 2 was preserved. Higher treatment pressures of process 4 could lead to an undesirable decrease of surface roughness especially for harder surface treatment media like alumina (see FIG. 6).

1. A process for producing a zirconia based dental implant, the process comprising the steps
providing a zirconia based dental implant precursor having a density of below about 4 g/cm³,
conducting a first surface treatment step to at least a part of the surface of the zirconia based dental implant precursor,
sintering the zirconia based dental implant precursor to obtain a sintered zirconia dental implant precursor,
conducting a second surface treatment step to at least those parts of the surface of the sintered zirconia based dental implant precursor, which have been surface treated before.

2. The process of claim 1, the zirconia based dental implant precursor comprising a metal oxide partially stabilized zirconia or an alumina toughened zirconia.

3. The process according to claim 1, the metals of the metal oxide partially stabilized zirconia being selected from Y, Mg, Ce, La, Ca, Er, Pr and mixtures thereof.

4. The process according to claim 1, the material the zirconia based dental implant precursor is made of comprising alumina in an amount from about 0.01 wt.-% to about 30 wt. %.

5. The process according to claim 1, the material the zirconia based dental implant precursor is made of being characterized in the presintered state by at least one of or all of the following features:
Biaxial flexural bending strength: 10 to 80 MPa according to EN ISO 6872:2008;
Density: below about 4.0 g/cm³;
Hardness: 30 to 70 HV1.

6. The process according to claim 1, wherein the first surface treatment step is done under conditions until a surface roughness Ra of the dental implant precursor of above about 1.2 μm is achieved.

7. The process according to claim 1, the first surface treatment step being conducted with a first surface treatment medium being characterized by at least one of or all of the following features:
comprising an inorganic material selected from zirconia, alumina and mixtures thereof or an organic material selected from polyvinylchloride, polystyrol, polyamide, nut shells, fruit stones, hard wood and mixtures thereof;
hardness: above about R100 (Rockwell hardness) for organic materials to above about HV1 2500 (Vickers hardness) for inorganic materials;
mean particle size: 50 to 250 μm.

8. The process according to claim 1, the sintering being done until the density of the zirconia based dental implant precursor is above 4.5 g/cm³

9. The process according to claim 1, the second surface treatment step being done under conditions to maintain a surface roughness Ra of the dental implant precursor of above about 1 μm.

10. The process according to claim 1, the second surface treatment step being conducted with a second surface treatment medium being characterized by at least one of the following features:
comprising a material selected from zirconia, alumina, boron carbide, boron nitride, tungsten carbide and mixtures thereof;
hardness: HV1≧1250;
mean particle size: 10 to 500 μm.

11. The process according to claim 1, wherein the zirconia based dental implant precursor is obtained by a process comprising
a machining step,
a casting or an injection molding step, or
a build-up technology.

12. The process according to claim 1,
the first surface treatment step being conducted under the following conditions: pressure: from about 1 to about 2 bar;
the sintering step being conducted under the following conditions: temperature: from about 1200 to about 1600° C.; duration: from about 1 to about 3 h; atmosphere: air
the second surface treatment step being conducted under the following conditions: pressure: from about 1.5 to about 4 bar.

13. A zirconia based dental implant obtainable according to a process as described in claim 1, the dental implant having a core region and a surface region,
the surface roughness Ra being above about 1 μm and
the crystal structure of the surface region being different from the crystal structure of the core region.

14. The zirconia based dental implant according to the preceding claim, the dental implant having at least two sections A and B, section A comprising at least about 60% of the surface of the dental implant, section B comprising less than about 40% of the surface of the dental implant, the surface of section A having been treated with the surface treatment steps as described in claim 1.

15. The zirconia based dental implant according to claim 12 being characterized by at least one of the following features:
Biaxial flexural bending strength: above about 850 MPa according to EN ISO 6872:2008;
Density: above about 4.5 g/cm³;
Monoclinic phase content within surface region measured down to a depth of about 5 μm: from about 0 to about 15%,
Distorted phase content within surface region measured down to a depth of about 5 μm: above about 20%.

16. A kit of parts comprising
at least one zirconia based dental milling block, especially a zirconia based dental milling block being suitable for being machined to a zirconia based dental implant precursor;
a first surface treatment medium as described in claim 7;
a second surface treatment medium as described in claim 10.
